(19) United States
(12) Patent Application Publication
(10) Pub. No.: US 2013/0330745 A1
(43) Pub. Date: Dec. 12, 2013

(54) METHODS OF PROGNOSIS AND DIAGNOSIS IN SEPSIS

(71) Applicant: Abbott Japan Co. Ltd., Chiba (JP)

(72) Inventor: Makota Komori, Chiba (JP)

(21) Appl. No.: 13/718,242

(22) Filed: Dec. 18, 2012

Related U.S. Application Data
(60) Provisional application No. 61/577,728, filed on Dec. 20, 2011.
(60) Provisional application No. 61/583,458, filed on Jan. 5, 2012.

(51) Int. Cl. G01N 33/543

(52) U.S. Cl. CPC G01N 33/543
USPC 435/7.92

(57) ABSTRACT

The present disclosure provides methods of diagnosing sepsis in patients by detecting the presence and amounts of biomarkers of sepsis in samples from the patients. Such biomarkers may be used to develop a more accurate prognosis for a patient with sepsis, or to accurately diagnose a patient suspected of having sepsis.

The subject application claims priority to U.S. provisional application No. 61/577,728 filed on Dec. 20, 2011 and U.S. provisional application No. 61/583,458 filed on Jan. 5, 2012, both of which are herein incorporated in their entirety by reference.

### TECHNICAL FIELD

The subject invention relates to methods for determining prognosis or risk stratification of sepsis in patients by detecting particular biomarkers in the patients as well as amounts thereof. Such biomarkers may be used to accurately develop a prognosis for a patient with sepsis or at risk of having sepsis, to stratify risk in a patient having sepsis, and also to develop a diagnosis of sepsis in a patient.

### BACKGROUND OF THE INVENTION

Sepsis is a disease defined as systemic inflammatory response syndrome (SIRS) with infectious disease. Despite improvements in supportive care, sepsis remains the most common cause of death in intensive care units, with mortality rates of 30% to 50%. Diagnosis of sepsis, such as by using blood markers, is desirable, because early treatment of sepsis can improve mortality. Several biomarkers have been proposed to diagnose sepsis, one of which is procalcitonin. However, the performance of sensitivity and specificity of those markers is not satisfactory. Therefore, there is a need in the art of new biomarkers that can be used to diagnose sepsis.

### SUMMARY OF THE INVENTION

In one aspect, the present disclosure relates to a method for providing a diagnosis, prognosis or risk classification of a subject having or at risk of having sepsis, the method comprising determining the concentration of angiopoietin-1 in a biological sample from the subject and comparing the determined angiopoietin-1 concentration with a reference angiopoietin-1 value, wherein a determined angiopoietin-1 concentration of the subject greater than the reference angiopoietin-1 value is indicative of sepsis or increased risk of sepsis in the subject. The method may further comprise assessing at least one additional biomarker of sepsis. In the method, providing a diagnosis can be providing a diagnosis of sepsis. Alternatively, providing a prognosis can be determining sepsis severity, or can be risk assessment of the subject with sepsis.

The method may further comprise the assessment of at least one additional biomarker of sepsis selected from the group consisting of angioppoietin-2 and procalcitonin. Assessment of the additional biomarker may comprise, for example, measuring the concentration of the biomarker in the biological sample from the subject, or may comprise a clinical evaluation of the subject. For an additional biomarker assessed by measuring the concentration of the biomarker in the biological sample from the subject, the method may further comprise comparing the measured concentration of the at least one further biomarker with a reference value for the biomarker. The reference value for the additional biomarker can be the biomarker concentration of a control sample, a biomarker cutoff value, or a median concentration of a plurality of control samples from a group of control subjects.

In any of the methods, the angiopoietin-1 reference value can be the angiopoietin-1 concentration of a control sample or an angiopoietin-1 cutoff value. The angiopoietin-1 concentration can be, for example, the angiopoietin-1 plasma or serum concentration. The control sample can be a biological sample of a control subject or an angiopoietin-1 standard. The angiopoietin-1 concentration of a control sample can be, for example, the median angiopoietin-1 concentration of a plurality of control samples from a group of control subjects (e.g., patients that do not have sepsis). Alternatively, an angiopoietin-1 cutoff value can be determined by a receiver operating curve (ROC) analysis from biological samples of a patient group. Alternatively, an angiopoietin-1 cutoff value can be determined by a quartile analysis of biological samples of a patient group. For example, an angiopoietin-1 cutoff value can be determined by selecting a value that corresponds to the median of a patient group consisting of patients with sepsis, which can be, for example, about 20 ng/ml serum. Alternatively, an angiopoietin-1 cutoff value can be determined by selecting a value that corresponds to the 75^{th} percentile of a patient group consisting of patients sepsis, which can be for example about 28 ng/mL serum. In other embodiments, an appropriate cutoff value can be about 2, about 3, about 4, about 5, about 6, about 7 about 8, about 9, about 10, about 11, about 12, about 13, about 14, about 15, about 16, about 17, about 18, about 19, about 21, about 22, about 23, about 24, about 25, about 26, about 27, about 29 or about 30 ng/mL serum. For example, a cutoff value of about 5 ng/mL serum may be used to discriminate sepsis specimens and normal specimens. Similar cutoff values can be used for blood plasma and blood samples.

In any of the methods, the subject can be a human subject and the biological sample of the subject and/or the control sample can be taken from a human subject. In any of the methods, the biological sample can be a bodily fluid, including any one of whole blood, plasma, serum, urine or any cell culture suspension or fraction of any thereof. In one aspect of the method, the sample is whole blood, plasma or serum, preferably plasma or serum. A coagulation inhibitor can be added to any peripheral blood sample. In the method, determining the concentration of angiopoietin-1, and optionally the at least one additional biomarker, can be performed by an immunological assay method in which a reagent capable of specific binding to angiopoietin-1, and optionally a reagent capable of specific binding to the additional biomarker, are used.

The present disclosure also provides a kit for performing any of the methods disclosed herein, wherein the kit includes at least one reagent capable of specifically binding angiopoietin-1, to quantify the angiopoietin-1 concentration in a biological sample of a subject, and a reference standard indicating a reference angiopoietin-1 concentration. In a kit for performing a method for providing a diagnosis, prognosis or risk classification of a subject having or at risk of having sepsis, the kit may further comprise at least one additional reagent capable of specifically binding at least one additional biomarker of sepsis in the biological sample, to quantify the concentration of the at least one additional biomarker in the biological sample, and also a reference standard indicating a reference concentration of the at least one additional biomarker of sepsis in the biological sample. In any of the kits, the at least one reagent capable of specifically binding angiopoietin-1 may comprise at least one antibody capable of specifically binding angiopoietin-1.

### BRIEF DESCRIPTION OF THE DRAWINGS

FIGS. 1A-1D are scatter plots of individual sepsis and normal samples tested according to Example 1.95% CI (Confidence Interval) Mean Diamond in each of FIGS. 1A-1D is calculated by the measured values of specimens.

FIG. 1A is a scatter plot of individual Angiopoietin-1 levels measured in the serum from sepsis specimens (left side) and normal individuals (right side).

FIG. 1B is a scatter plot of individual Angiopoietin-2 levels measured in the serum from sepsis specimens (left side) and normal individuals (right side).

FIG. 1C is a scatter plot of individual levels of Procalcitonin by Roche Brahms ECLusys measured in the serum from sepsis specimens (left side) and normal individuals (right side).

FIG. 1D is a scatter plot of individual levels of Procalcitonin by the ELISA of Ray Biotech Co., Ltd. measured in the serum from sepsis specimens (left side) and normal individuals (right side).

FIG. 2 is a Receiver Operating Characteristic (ROC) plot of Procalcitonin, Angiopoietin-1, Angiopoietin-2.

FIGS. 3A-3D are scatter plots of individual values of Angiopoietin-1 and Angiopoietin-2 by the R&D Systems ELISA kit and the Ray Biotech ELISA kit.

FIG. 3A is a scatter plot of individual levels of Angiopoietin-1 by the ELISA of R&D Systems measured in the serum from sepsis specimens (left side) and normal individuals (right side).

FIG. 3B is a scatter plot of individual levels of Angiopoietin-1 by the ELISA of Ray Biotech Co., Ltd. measured in the serum from sepsis specimens (left side) and normal individuals (right side).

FIG. 3C is a scatter plot of individual levels of Angiopoietin-2 by the ELISA of R&D Systems measured in the serum from sepsis specimens (left side) and normal individuals (right side).

FIG. 3D is a scatter plot of individual levels of Angiopoietin-2 by the ELISA of Ray Biotech Co., Ltd. as measured in the serum from sepsis specimens (left side) and normal individuals (right side).

FIG. 4 is a Receiver Operating Characteristic (ROC) plot of Angiopoietin-1 and Angiopoietin-2 by the R&D Systems ELISA kit and the Ray Biotech ELISA kit.

### DETAILED DESCRIPTION

The present disclosure is based on the unexpected discovery of a strong and independent association between angiopoietin-1 and sepsis. In particular, the present disclosure discloses for the first time an association between higher angiopoietin-1 levels, and the incidence of sepsis. As described herein, angiopoietin-1 is significantly associated with sepsis. The association between angiopoietin-1 and sepsis is robust, predictive of clinical outcomes in sepsis, and, survives even after adjusting for numerous confounding variables. The assessment of angiopoietin-1 can improve on the current ability to stratify patient risk and develop a prognosis in patients, thereby significantly benefiting patients having or at risk of developing sepsis. Further, the combined use of angiopoietin-1 and additional biomarkers can provide comparable advantages.

Accordingly, the present disclosure provides methods of providing a diagnosis, prognosis or risk classification of a subject or group of subjects having or at risk of having sepsis, using angiopoietin-1 as a novel clinical biomarker of adverse outcomes. Also provided are kits for performing the disclosed methods.

Section headings as used in this section and the entire disclosure herein are not intended to be limiting.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the term “including”, as well as other forms, such as “includes” and “included”, is not limiting.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

“Control” as used herein when referring to a composition, refers to a composition known to not contain an analyte of interest (“negative”), e.g., angiopoietin (such as angiopoietin-1, angiopoietin-2, fragments of angiopoietin-1, fragments of angiopoietin-2, variants of angiopoietin (angiopoietin-1 or angiopoietin-2) or any combinations thereof), procalcitonin, or any combinations thereof, or to contain an analyte of interest (“positive control”), e.g., angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), procalcitonin or any combinations thereof. A positive control can comprise a known concentration of angiopoietin or procalcitonin. “Control,” “positive control,” and “calibrator” may be used interchangeably herein to refer to a composition comprising a known concentration of angiopoietin or procalcitonin. A “positive control” can be used to establish assay performance characteristics and is a useful indicator of the integrity of reagents (e.g., analytes). A “normal control” may refer to a sample or a subject that does not have sepsis, or is not at risk of having sepsis.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%, or at least about 40%, at least about 45%, at least about 50%, at least about 55%, at least about 60%, at least about 65%, at least about 70%, at least about 75%, at least about 80%, at least about 85%, at least about 90%, or at least about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). The present disclosure provides exemplary predetermined levels. However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations as described herein should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), procalcitonin, or any combinations thereof) entails release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Sample,” “test sample,” and “patient sample” may be used interchangeably herein. The sample, such as a sample of urine, serum, plasma, amniotic fluid, cerebrospinal fluid, placental cells or tissue, endothelial cells, leukocytes, or monocytes, can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“As used herein, the term “angiopoietin-1” refers to the circulating form of the angiopoietin-1 growth factor, which is a secreted glycoprotein with an affinity for an endothelial cell-specific tyrosine-protein kinase receptor.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Tracer” as used herein refers to an analyte or analyte fragment conjugated to a label, such as angiopoietin-1 conjugated to a fluorescein moiety, wherein the analyte conjugated to the label can effectively compete with the analyte for sites on an antibody specific for the analyte.

As used herein, the term “sepsis” refers to a condition in which a systemic inflammatory response syndrome (SIRS) occurs in the presence of a known or suspected infection. The inflammatory response by the immune system can occur in response to microbes in the blood, urine, lungs, skin, or other tissues.

The American College of Chest Physicians and the Society of Critical Care Medicine describe different levels of sepsis. Systemic inflammatory response syndrome (SIRS) which occurs in sepsis is defined by the presence of two or more of the following findings: (i) Body temperature less than 36° C. (97° F.) or more than 38° C. (100° F.) (hypothermia or fever); (ii) Heart rate more than 90 beats per minute; (iii) Respiratory rate more than 20 breaths per minute or, on blood gas, a PₐCO₂ less than 32 mm Hg (4.3 kPa) (tachypnea or hypocapnia due to hyperventilation); (iv) White blood cell count less than 4,000 cells/mm³ or more than 12,000 cells/mm³ (less than 4×109 or more than 12×109 cells/L), or greater than 10% band forms (immature white blood cells). Sepsis is when SIRS occurs in the presence of a known or suspected infection. Severe sepsis is sepsis with organ dysfunction, hypoperfusion, or hypotension.

Septic shock occurs when there is sepsis with refractory arterial hypotension or hypoperfusion abnormalities in spite of adequate fluid resuscitation. Signs of systemic hypoperfusion may be either end-organ dysfunction or serum lactate greater than 4 mmol/L. Other signs include oliguria and altered mental status. Patients are defined as having septic shock if they have sepsis plus hypotension after aggressive fluid resuscitation (typically upwards of 6 liters or 40 ml/kg of crystalloid solution).

Examples of end-organ dysfunction include the following: acute lung injury (ALI) (PaO2/FiO2<300) or acute respiratory distress syndrome (ARDS) (PₐO₂/FiO₂<200); encephalopathy, with symptoms of agitation, confusion, coma, caused from ischemia, hemorrhage, microthrombi, microabscesses, or multifocal necrotizing leukoencephalopathy; disruption of protein synthetic function in liver, manifesting acutely as progressive coagulopathy due to inability to synthesize clotting factors; disruption of liver metabolic functions, manifesting as cessation of bilirubin metabolism, resulting in elevated unconjugated serum bilirubin levels (indirect bilirubin); oliguria and anuria in kidney, electrolyte abnormalities, volume overload; systolic and diastolic heart failure, likely due to cytokines that depress myocyte function; cellular damage of heart, manifesting as a troponin leak (although not necessarily ischemic in nature).

As used herein, the terms “risk assessment” or “risk stratification” of subjects refers to the evaluation of factors including biomarkers, to predict the risk of occurrence of future events including death, so that treatment decisions regarding the subject may be made on a more informed basis.

As used herein, the term “sepsis risk” of subjects refers to the evaluation of factors including biomarkers, to predict the risk of occurrence of sepsis including increased probability of sepsis, and death due to sepsis.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). The subject may be a human or a non-human.

The subject may be a critical care patient, undergoing chemotherapy, recovering from surgery, or may be at risk for, or have, an infection, cancer, an autoimmunce disease or disorder, chronic organ disease and/or inflammation, and/or chronic rejection of an organ after solid organ transplantation. The infection may be acute or chronic. The infection may be viral, bacterial, parasitic, or fungal. The cancer may be any cancer, such as hematologic or a solid tumor. The autoimmune disease may be any autoimmune disease, such as rheumatoid arthritis, systemic lupus erythematosus and connective tissue diseases, vasculitis, sarcoidosis, and inflammatory bowel disease. The chronic organ disease may be chronic kidney disease, in which the subject may or may not be undergoing dialysis. The viral infection may be hepatitis B or C infection, or human immunodeficiency virus infection. Any of the diseases and disorders may be an underlying cause of sepsis. The surgery may be perioperative or postoperative. The surgery may be oncologic surgery.

The terms “sample” and “biological sample” as used herein generally refer to a biological material being tested for and/or suspected of containing an analyte of interest such as angiopoietin-1. The sample may be any tissue sample from the subject. The sample may comprise protein from the subject.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein purification may not be necessary.

Methods well-known in the art for collecting, handling and processing urine, blood, serum and plasma, and other body fluids, are used in the practice of the present disclosure, for instance, when the antibodies provided herein are employed as immunodiagnostic reagents, and/or in an angiopoietin immunoassay kit. The test sample can comprise further moieties in addition to the angiopoietin analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples), pretreatment optionally can be done for mere convenience (e.g., as part of a regimen on a commercial platform). The sample may be used directly as obtained from the subject or following pretreatment to modify a characteristic of the sample. Pretreatment may include extraction, concentration, inactivation of interfering components, and/or the addition of reagents.

The pretreatment reagent can be any reagent appropriate for use with the assay, e.g., immunoassay, and kit described herein. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, European Pat. Pub. No. 0 471 293, and U.S. Pat. App. Pub. No. 2008/0020401 (incorporated by reference in its entirety for its teachings regarding pretreatment). The pretreatment reagent can be a heterogeneous agent or a homogeneous agent.

With use of a heterogeneous pretreatment reagent, the pretreatment reagent precipitates analyte binding protein (e.g., protein that can bind to angiopoietin (angiopoietin-1 or angiopoietin-2) or a fragment thereof) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner for angiopoietin (angiopoietin-1 or angiopoietin-2), variants of angiopoietin (angiopoietin-1 or angiopoietin-2) such as a labeled anti-angiopoietin monoclonal antibody (or an antigenically reactive fragment thereof). The pretreatment reagent employed for such an assay typically is diluted in the pretreated test sample mixture, either before or during capture by the first specific binding partner. Despite such dilution, a certain amount of the pretreatment reagent (for example, 5 M methanol and/or 0.6 Methylene glycol) is still present (or remains) in the test sample mixture during capture.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHODS

The methods encompass providing a prognosis of a subject which includes, with respect to sepsis, any one or more of determining the severity of sepsis, determining the subject's risk for subsequent all-cause mortality, and risk assessment of the subject with sepsis. The methods are based in part on the novel finding that angiopoietin, particularly, angiopoietin-1 concentration in a biological sample from a subject with sepsis predicts adverse outcomes of the subject, and thus that angiopoietin-1 is a prognosis marker for sepsis. For example, the angiopoietin-1 concentration of a subject can be used to provide a prognosis with respect to the risk for subsequent all-cause mortality, or death, such as, for example, a mortality prediction within a given number of days, weeks or months.

The methods involve providing or obtaining a biological sample from the subject, which can be obtained by any known means including needle stick, needle biopsy, swab, and the like. In one aspect, the biological sample is a blood sample, preferably a blood plasma or serum sample, which may be obtained for example by venipuncture. Biological samples may be or have been stored or banked under suitable tissue storage conditions.

The methods encompass a method for diagnosis, prognosis and/or risk stratification of sepsis in a subject having or suspected of having sepsis by determining an angiopoietin-1 concentration in the subject. Providing a diagnosis can be, for example, providing a diagnosis of sepsis. Providing a prognosis can be, for example, determining sepsis severity, or can be a risk assessment, i.e. determination of risk of sepsis of the subject. The methods also encompass identifying one or more patients or a subgroup of patients having an increased risk of sepsis. A shared feature of all methods is the determination of concentration of angiopoietin-1 in a biological sample as described herein, wherein an increased concentration of angiopoietin-1 in the sample relative to a reference value for angiopoietin-1 concentration is indicative of sepsis, or increased risk of sepsis.

The angiopoietin-1 concentration is deemed increased in comparison to a reference value, i.e. the angiopoietin-1 reference value as described herein. For example, an angiopoietin-1 serum concentration useful as a reference value is about 5 ng/ml, but can be higher or lower, for example about 3 or 10 ng/ml in serum. The angiopoietin-1 concentration may be deemed increased as compared to the reference value when it is significantly higher, e.g. at least 20% higher (1.2 fold), at least 30% (1.3 fold) higher, at least 40% higher (1.4 fold), at least 50% higher (1.5 fold), at least 60% higher (1.6 fold), at least 70% higher (1.7 fold), at least 80% higher (1.8 fold), at least 100% higher (2.0 fold or double), at least 150% higher (2.5 fold), or at least 200% higher (3.0 fold or triple). The reference value or angiopoietin-1 reference value can be used as predetermined cutoff or predetermined level in methods described herein.

The presence, concentration or amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) present in a body sample may be readily determined using any suitable assay as is known in the art. Examples include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay, a capture reagent that specifically binds an angiopoietin (or a fragment thereof) of interest is attached to the surface of a mass spectrometry probe, such as a pre-activated protein chip array. The angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) is then specifically captured on the biochip, and the captured angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) is detected by mass spectrometry. Alternatively, the angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) can be eluted from the capture reagent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an example of an immunoassay that can be used. Other methods include, for example, mass spectrometry and immunohistochemistry (e.g. with sections from tissue biopsies) using antibodies (such as monoclonal, polyclonal, chimeric, humanized, human antibodies, etc.) or fragments thereof against angiopoietin. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

Angiopoietin, and/or peptides or fragments thereof (such as angiopoietin-1, angiopoietin-2, fragments of angiopoietin-1, fragments of angiopoietin-2, variants of angiopoietin (angiopoietin-1 or soluble angiopoietin-2) or any combinations thereof), may be analyzed using an immunoassay. The presence or amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) can be determined using the herein-described antibodies and detecting specific binding to angiopoietin. If desired, one or more of the antibodies described herein can be used in combination with one or more commercially available monoclonal/polyclonal antibodies. Alternatively, commercially available antibodies can solely be used in said assays. Such commercially available antibodies are available from companies such as R&D Systems, Inc. (Minneapolis, Minn.), Abcam (Cambridge, Mass.) and Enzo Life Sciences International, Inc. (Plymouth Meeting, Pa.).

Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA.

A heterogeneous format may be used. For example, after the test sample is obtained from a subject, a first mixture is prepared. The mixture contains the test sample being assessed for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) and a first specific binding partner, wherein the first specific binding partner and any angiopoietin contained in the test sample form a first specific binding partner-angiopoietin complex. Preferably, the first specific binding partner is an anti-angiopoietin antibody or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-angiopoietin complex is formed, any unbound angiopoietin is removed from the complex using any technique known in the art. For example, the unbound angiopoietin can be removed by washing. Desirably, however, the first specific binding partner is present in excess of any angiopoietin present in the test sample, such that all angiopoietin that is present in the test sample is bound by the first specific binding partner.

After any unbound angiopoietin is removed, a second specific binding partner is added to the mixture to form a first specific binding partner-angiopoietin-second specific binding partner complex. The second specific binding partner is preferably an anti-angiopoietin antibody that binds to an epitope on angiopoietin that differs from the epitope on angiopoietin bound by the first specific binding partner. Moreover, also preferably, the second specific binding partner is labeled with or contains a detectable label as described above.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The Sandwich ELISA measures the amount of antigen between two layers of antibodies (i.e., a capture antibody (i.e., at least one capture antibody) and a detection antibody (i.e. at least one detection antibody). The capture antibody and the detection antibody bind to different epitopes on the antigen, e.g., angiopoietin. Desirably, binding of the capture antibody to an epitope does not interfere with binding of the detection antibody to an epitope. Either monoclonal or polyclonal antibodies may be used as the capture and detection antibodies in the sandwich ELISA.

Generally, at least two antibodies are employed to separate and quantify angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in a test sample. More specifically, the at least two antibodies bind to certain epitopes of angiopoietin or an angiopoietin fragment forming an immune complex which is referred to as a “sandwich”. One or more antibodies can be used to capture the angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. In other words, antibodies are selected so that the one or more first antibodies brought into contact with a test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) do not bind to all or part of an epitope recognized by the second or subsequent antibodies, thereby interfering with the ability of the one or more second detection antibodies to bind to the angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof).

An example of an antibody that can be used as a first antibody in said immunoassay are antibodies that immunospecifically bind to an epitope comprising at least three contiguous (3) amino acids of angiopoietin (such as angiopoietin-1 or angiopoietin-2) with a K_{D} of from 4.2×10⁻¹¹ M to 7.4×10⁻¹³ M. The immunoassay may comprise a second antibody that immunospecifically binds to an epitope comprising at least three contiguous (3) amino acids of angiopoietin (such as angiopoietin-1 or angiopoietin-2), wherein the contiguous (3) amino acids to which the second antibody binds is different from the three (3) contiguous amino acids to which the first antibody binds. The second antibody can also have a K_{D} of from 4.2×10⁻¹¹ M to 7.4×10⁻¹³ M.

In a preferred embodiment, a test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) can be contacted with at least one capture antibody (or antibodies) and at least one detection antibodies either simultaneously or sequentially. In the sandwich assay format, a test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) is first brought into contact with the at least one capture antibody that specifically binds to a particular epitope under conditions which allow the formation of a antibody-angiopoietin complex. If more than one capture antibody is used, a multiple capture antibody-angiopoietin complex is formed. In a sandwich assay, the antibodies, preferably, the at least one capture antibody, are used in molar excess amounts of the maximum amount of angiopoietin or the angiopoietin fragment expected in the test sample. For example, from about 5 μg/mL to about 1 mg/mL of antibody per mL of microparticle coating buffer may be used.

Optionally, prior to contacting the test sample with the at least one first capture antibody, the at least one capture antibody can be bound to a solid support which facilitates the separation the antibody-angiopoietin complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind angiopoietin or angiopoietin fragment. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) is brought into contact with the at least one capture antibody, the test sample is incubated in order to allow for the formation of a capture antibody (or capture antibodies)-angiopoietin complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the capture antibody (antibodies)-angiopoietin complex, the complex is then contacted with at least one detection antibody (under conditions which allow for the formation of a capture antibody (antibodies)-angiopoietin-detection antibody (antibodies) complex). If the capture antibody-angiopoietin complex is contacted with more than one detection antibody, then a capture antibody (antibodies)-angiopoietin-detection antibody (antibodies) detection complex is formed. As with the capture antibody, when the at least one detection (and subsequent) antibody is brought into contact with the capture antibody-angiopoietin complex, a period of incubation under conditions similar to those described above is required for the formation of the capture antibody (antibodies)-angipoietin-detection antibody (antibodies) complex. Preferably, at least one detection antibody contains a detectable label. The detectable label can be bound to the at least one detection antibody prior to, simultaneously with or after the formation of the capture antibody (antibodies)-angiopoietin-detection antibody (antibodies) complex. Any detectable label known in the art can be used as discussed herein and known in the art.

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-angiopoietin complexes form. Alternatively, if a second specific binding partner is used and the second specific binding partner is detectably labeled with an acridinium compound, detectably labeled first specific binding partner-angiopoietin-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of angiopoietin or a fragment thereof is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the sample can be quantified. Specifically, the amount of angiopoietin in the sample is proportional to the intensity of the signal generated. The amount of angiopoietin present can be quantified by comparing the amount of light generated to a standard curve for angiopoietin or by comparison to a reference standard. The standard curve can be generated using serial dilutions or solutions of known concentrations of angiopoietin by mass spectroscopy, gravimetric methods, and other techniques known in the art.

In a chemiluminescent microparticle assay employing the ARCHITECT® (or its successor) analyzer, the conjugate diluent pH should be about 6.0+/−0.2, the microparticle coating buffer should be maintained at room temperature (i.e., at about 17 to about 27° C.), the microparticle coating buffer pH should be about 6.5+/−0.2, and the microparticle diluent pH should be about 7.8+/−0.2. Solids preferably are less than about 0.2%, such as less than about 0.15%, less than about 0.14%, less than about 0.13%, less than about 0.12%, or less than about 0.11%, such as about 0.10%.

In a forward competitive format, an aliquot of labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) of a known concentration is used to compete with angiopoietin in a test sample for binding to angiopoietin antibody (such as an antibody).

In a forward competition assay, an immobilized antibody (such as an antibody) can either be sequentially or simultaneously contacted with the test sample and a labeled angiopoietin, angiopoietin fragment or angiopoietin variant thereof. The angiopoietin peptide, angiopoietin fragment or angiopoietin variant can be labeled with any detectable label, including those detectable labels discussed above in connection with the anti-angiopoietin antibodies. In this assay, the antibody can be immobilized on to a solid support. Alternatively, the antibody can be coupled to an antibody, such as an antispecies antibody, that has been immobilized on a solid support, such as a microparticle.

The labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), the test sample and the antibody are incubated under conditions similar to those described above in connection with the sandwich assay format. Two different species of antibody-angiopoietin complexes may then be generated. Specifically, one of the antibody-angiopoietin complexes generated contains a detectable label while the other antibody-angiopoietin complex does not contain a detectable label. The antibody-angiopoietin complex can be, but does not have to be, separated from the remainder of the test sample prior to quantification of the detectable label. Regardless of whether the antibody-angiopoietin complex is separated from the remainder of the test sample, the amount of detectable label in the antibody-angiopoietin complex is then quantified. The concentration of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample can then be determined by comparing the quantity of detectable label in the antibody-angiopoietin complex to a standard curve. The standard curve can be generated using serial dilutions of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) of known concentration, by mass spectroscopy, gravimetrically and by other techniques known in the art.

The antibody-angiopoietin complex can be separated from the test sample by binding the antibody to a solid support, such as the solid supports discussed above in connection with the sandwich assay format, and then removing the remainder of the test sample from contact with the solid support.

In a reverse competition assay, an immobilized angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) can either be sequentially or simultaneously contacted with a test sample and at least one labeled antibody. Preferably, the antibody specifically binds to an epitope comprising at least 3, at least 4, at least 5, at least 6, at least 7, at least 8, at least 9, or at least 10, at least 15, at least 20, at least 25 or at least 30 amino acids of angiopoietin (such as angiopoietin-1 or angiopoietin-2).

The angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) can be bound to a solid support, such as the solid supports discussed above in connection with the sandwich assay format. Preferably, the angiopoietin (angiopoietin-1 or angiopoietin-2) peptide fragment comprises at least 3, at least 4, at least 5, at least 6, at least 7, at least 8, at least 9, at least 10, at least 15, at least 20, at least 25 or at least 30 amino acids.

The immobilized angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), test sample and at least one labeled antibody are incubated under conditions similar to those described above in connection with the sandwich assay format. Two different species angiopoietin-antibody complexes are then generated. Specifically, one of the angiopoietin-antibody complexes generated is immobilized and contains a detectable label while the other angiopoietin-antibody complex is not immobilized and contains a detectable label. The non-immobilized angiopoietin-antibody complex and the remainder of the test sample are removed from the presence of the immobilized angiopoietin-antibody complex through techniques known in the art, such as washing. Once the non-immobilized angiopoietin antibody complex is removed, the amount of detectable label in the immobilized angiopoietin-antibody complex is then quantified. The concentration of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample can then be determined by comparing the quantity of detectable label in the angiopoietin-complex to a standard curve. The standard curve can be generated using serial dilutions of angiopoietin or angiopoietin fragment of known concentration, by mass spectroscopy, gravimetrically and by other techniques known in the art.

In a fluorescence polarization assay, an antibody or functionally active fragment thereof may be first contacted with an unlabeled test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) to form an unlabeled angiopoietin-antibody complex. The unlabeled angiopoietin-antibody complex is then contacted with a fluorescently labeled angiopoietin (such as angiopoietin-1, angiopoietin-2, fragments of angiopoietin-1, fragments of angiopoietin-2, variants of angiopoietin (angiopoietin-1 or soluble angiopoietin-2). The labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) competes with any unlabeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample for binding to the antibody or functionally active fragment thereof. The amount of labeled angiopoietin-antibody complex formed is determined and the amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample determined via use of a standard curve.

The antibody used in a fluorescence polarization assay specifically binds to an epitope comprising at least 3, at least 4, at least 5, at least 6, at least 7, at least 8, at least 9, at least 10, at least 15, at least 20, at least 25 or at least 30 amino acids of angiopoietin (such as angiopoietin-1 or angiopoietin-2).

The antibody, labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) and test sample and at least one labeled antibody may be incubated under conditions similar to those described above in connection with the sandwich immunoassay.

Alternatively, an antibody or functionally active fragment thereof may be simultaneously contacted with a fluorescently labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) and an unlabeled test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) to form both labeled angioipoietin-antibody complexes and unlabeled angiopoietin-antibody complexes. The amount of labeled angiopoietin-antibody complex formed is determined and the amount of angiopoietin in the test sample determined via use of a standard curve. The antibody used in this immunoassay specifically may bind to an epitope comprising at least 3, at least 4, at least 5, at least 6, at least 7, at least 8, at least 9, at least 10, at least 15, at least 20, at least 25 or at least 30 amino acids of angiopoietin.

Alternatively, an antibody or functionally active fragment thereof is first contacted with a fluorescently labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) to form a labeled angiopoietin-antibody complex. The labeled angiopoietin-antibody complex is then contacted with an unlabeled test sample suspected of containing angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof). Any unlabeled angiopoietin in the test sample competes with the labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) for binding to the antibody or functionally active fragment thereof. The amount of labeled angiopoietin-antibody complex formed is used to determine the amount of angiopoietin in the test sample via use of a standard curve. The antibody used in this immunoassay specifically binds to an epitope comprising at least three 3, at least 4, at least 5, at least 6, at least 7, at least 8, at least 9, at least 10, at least 15, at least 20, at least 25, or at least 30 amino acids of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof)

Mass spectrometry (MS) analysis may be used alone or in combination with other methods. Other methods include immunoassays and those described above to detect specific polynucleotides. The mass spectrometry method may be used to determine the presence and/or quantity of one or more biomarkers. MS analysis may comprise matrix-assisted laser desorption/ionization (MALDI) time-of-flight (TOF) MS analysis, such as, for example, directed—spot MALDI-TOF or liquid chromatography MALDI-TOF analysis. In some embodiments, the MS analsyis comprises electrospray ionization (ESI) MS, such as liquid chromatography (LC) ESI-MS. Mass analysis can be accomplished using commercially available spectrometers. Methods for utilizing MS analysis, including MALDI-TOF MS and ESI-MS, to detect the presence and quantity of biomarker peptides in biological samples may be used. See, for example, U.S. Pat. Nos. 6,925,389; 6,989,100; and 6,890,763 for guidance, each of which is incorporated herein by reference.

It may be desirable to include a control sample or a calibrator, such as a series of calibrators. The control sample may be analyzed concurrently with the sample from the subject as described above. The results obtained from the subject sample can be compared to the results obtained from the control sample. Standard curves may be provided, with which assay results for the biological sample may be compared. Such standard curves present levels as a function of assay units, i.e. fluorescent signal intensity, if a fluorescent label is used. Using samples taken from multiple donors, standard curves can be provided for control levels of the angiopoietin in normal tissue, as well as for “at-risk” levels of the angiopoietin in tissue taken from donors, who may have one or more of the characteristics set forth above.

Thus, in view of the above, a method of determining the presence, amount or concentration of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in a test sample is provided. The method comprises assaying the test sample for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) by an immunoassay, for example, employing at least one antibody and at least one detectable label and comprising comparing a signal generated by the detectable label as a direct or indirect indication of the presence, amount or concentration of angiopoietin in the test sample to a signal generated as a direct or indirect indication of the presence, amount or concentration of angiopoietin in a calibrator. The calibrator is optionally, and is preferably, part of a series of calibrators in which each of the calibrators differs from the other calibrators in the series by the concentration of angiopoietin. One of the at least one antibody is an isolated antibody, which specifically binds to angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), wherein the antibody has a domain or region selected from (i) a variably heavy domain region, or (ii) a variably heavy domain region and a variable light domain region. Alternatively, one of the at least one antibody is an isolated antibody, which specifically binds to angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), wherein the antibody has (i) a variable heavy chain comprising a complementarity determining region (CDR)1, a CDR2, and a CDR3 and a variable light chain comprising a CDR1, a CDR2, and a CDR3.

The method can comprise (i) contacting the test sample with at least one capture antibody, which binds to an epitope on angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), so as to form a capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex, (ii) contacting the capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex with at least one detection antibody, which comprises a detectable label and binds to an epitope on angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) that is not bound by the capture antibody, to form a capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof)/detection antibody complex, and (iii) determining the amount of angiopoietin (or a fragment thereof) in the test sample based on the signal generated by the detectable label in the capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof)/detection antibody complex formed in (ii).

Alternatively, the method can comprise (i) contacting the test sample with at least one capture antibody, which binds to an epitope angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) so as to form a capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex, and simultaneously or sequentially, in either order, contacting the test sample with detectably labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), which can compete with any angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) in the test sample for binding to the at least one capture antibody. Any angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) present in the test sample and the detectably labeled angiopoietin compete with each other to form a capture antibody/angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex and a capture antibody/detectably labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex, respectively. The method further comprises (ii) determining the presence, amount or concentration of angiopoietin in the test sample based on the signal generated by the detectable label in the capture antibody/detectably labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex formed in (ii). The signal generated by the detectable label in the capture antibody/detectably labeled angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) complex is inversely proportional to the amount or concentration of angiopoietin in the test sample.

In one embodiment, a mouse anti-angiopoietin Ab (such as an anti-angiopoietin-1 Ab) can be attached directly or indirectly, e.g., via a sheep (or other species) anti-mouse Ab, to a solid support. Any angiopoietin (such as angiopoietin-1), which is present in a sample and brought into contact with the solid support, is bound by the mouse anti-angiopoietin Ab. A biotin-labeled goat anti-angiopoietin Ab also binds to the angiopoietin. Streptavidin, which is linked to horseradish peroxidase (HRPO), binds to the biotin on the goat anti-angiopoietin Ab. Upon being contacted with o-phenylenediamine, the HRPO converts the o-phenylenediamine to 2,3-diaminophenazine, which is orange-brown in color and can be measured spectrophotometrically at 492 nm.

The method can further comprise diagnosing, prognosticating, or assessing the efficacy of a therapeutic/prophylactic treatment of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy. The method can be adapted for use in an automated system or a semi-automated system.

Generally, a predetermined level can be employed as a benchmark against which to assess results obtained upon assaying a test sample for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof). Generally, in making such a comparison, the predetermined level is obtained by running a particular assay a sufficient number of times and under appropriate conditions such that a linkage or association of analyte presence, amount or concentration with a particular stage or endpoint of a disease, disorder or condition (e.g. sepsis, or increased risk of sepsis) or with particular indicia can be made. Typically, the predetermined level is obtained with assays of reference subjects (or populations of subjects). The angiopoietin measured can include fragments thereof, degradation products thereof, and/or enzymatic cleavage products thereof.

In particular, with respect to a predetermined level as employed for monitoring disease progression and/or treatment, the amount or concentration of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for angiopoietin is defined in accordance with standard practice. A so-called altered level or alteration can be considered to have occurred when there is any net change as compared to the typical or normal level or range, or reference level or range that cannot be explained by experimental error or sample variation. Thus, the level measured in a particular sample will be compared with the level or range of levels determined in similar samples from a so-called normal subject. In this context, a “normal subject” is an individual with no detectable disease or disorder, and a “normal” (sometimes termed “control”) patient or population is/are one(s) that exhibit(s) no detectable disease or disorder, respectively, for example. An “apparently normal subject” is one in which angiopoietin has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, sepsis.

The method of assay can also involve the assay of other markers and the like as discussed herein and known in the art. For example, the method of assay can also involve the assay of angiopoietin-1, angiopoietin-2, and/or procalcitonin, for example.

The methods described herein also can be used to determine whether or not a subject has or is at risk of developing sepsis, such as discussed herein and known in the art. Specifically, such a method can comprise the steps of:

(a) determining the concentration or amount in a test sample from a subject of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) or any combinations thereof using the methods described herein, or methods known in the art); and

(b) comparing the concentration or amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) determined in step (a) with a predetermined level, wherein, if the concentration or amount of angiopoietin determined in step (a) is favorable with respect to a predetermined level, then the subject is determined not to have or be at risk for sepsis as discussed herein and known in in the art. However, if the concentration or amount of angiopoietin determined in step (a) is unfavorable with respect to the predetermined level, then the subject is determined to have or be at risk for sepsis as discussed herein and known in the art.

Additionally, provided herein is method of monitoring the progression of disease in a subject. Optimally, the method comprises the steps of:

(a) determining the concentration or amount in a test sample from a subject of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof);

(b) determining the concentration or amount in a later test sample from the subject of angiopoietin; and

(c) comparing the concentration or amount of angiopoietin as determined in step (b) with the concentration or amount of angiopoietin determined in step (a), wherein if the concentration or amount determined in step (b) is unchanged or is unfavorable when compared to the concentration or amount of angiopoietin determined in step (a), then the disease in the subject is determined to have continued, progressed or worsened. By comparison, if the concentration or amount of angiopoietin as determined in step (b) is favorable when compared to the concentration or amount of angiopoietin as determined in step (a), then the disease in the subject is determined to have discontinued, regressed or improved.

Optionally, the method further comprises comparing the concentration or amount of angiopoietin as determined in step (b), for example, with a predetermined level. Further, optionally the method comprises treating the subject with one or more pharmaceutical compositions for a period of time if the comparison shows that the concentration or amount of angiopoietin as determined in step (b), for example, is unfavorably altered with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions, such as one or more antibiotics. Next, the concentration or amount in a first test sample from a subject of angiopoietin is determined (e.g., using the methods described herein or as known in the art). After the concentration or amount of angiopoietin is determined, optionally the concentration or amount of angiopoietin is then compared with a predetermined level. If the concentration or amount of angiopoietin as determined in the first test sample is lower than the predetermined level, then the subject is not treated with one or more pharmaceutical compositions. However, if the concentration or amount of angiopoietin as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about one (1) day to about thirty (30) days, preferably from about three (3) days to about fourteen (14) days).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) or any combinations thereof) is determined in the second or subsequent test sample is determined (e.g., using the methods described herein or as known in the art). The concentration or amount of angiopoietin as determined in each of the second and subsequent test samples is then compared with the concentration or amount of angiopoietin as determined in the first test sample (e.g., the test sample that was originally optionally compared to the predetermined level). If the concentration or amount of angiopoietin as determined in step (c) is favorable when compared to the concentration or amount of angiopoietin as determined in step (a), then the disease or infection in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of angiopoietin as determined in step (a), then the disease or infection in the subject is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received to decrease or lower said subject's angiopoietin level.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years after the first test sample from the subject is obtained, or at least about after one of the aforementioned time periods. When used to monitor disease progression, the above assay can be used to monitor the progression of disease in subjects suffering from acute conditions. Acute conditions, also known as critical care conditions, refer to acute, life-threatening diseases or other critical medical conditions involving, for example, the cardiovascular system or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., repeated every about 1 minute, about 5 minutes, about 10 minutes, about 15 minutes, about 30 minutes, about 45 minutes, about 60 minutes, about 2 hours, about 3 hours, about 4 hours, 4 about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days, or at least about every one of the aforementioned timeframes, and the initial assay likewise is generally done within a shorter timeframe, e.g., about minutes, hours or days of the onset of the disease or condition.

The assays also can be used to monitor the progression of disease in subjects suffering from chronic or non-acute conditions. Non-critical care or, non-acute conditions, refers to conditions other than acute, life-threatening disease or other critical medical conditions involving, for example, the cardiovascular system and/or excretory system. Typically, non-acute conditions include those of longer-term or chronic duration. For non-acute conditions, repeat monitoring generally is done with a longer timeframe, e.g., hours, days, weeks, months or years (e.g., after about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years, or after at least about one of the aforementioned timeframes), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as urine, serum or plasma. Optionally the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a disease (e.g., sepsis, as discussed herein and known in the art) will benefit from treatment. In particular, the disclosure relates to angiopoietin companion diagnostic methods and products. Thus, the method of “monitoring the treatment of disease in a subject” as described herein further optimally also can encompass selecting or identifying candidates for therapy, such as therapy with antibiotics.

Thus, in particular embodiments, the disclosure also provides a method of determining whether a subject having, or at risk for, sepsis, as discussed herein and known in the art) is a candidate for therapy. Generally, the subject is one who has experienced some symptom of the disease or who has actually been diagnosed as having, or being at risk for, such a disease, and/or who demonstrates an unfavorable concentration or amount of angiopoietin or a fragment thereof, as described herein.

The method optionally comprises an assay as described herein, where analyte is assessed before and following treatment of a subject with one or more pharmaceutical compositions (e.g., particularly with a pharmaceutical related to a mechanism of action involving angiopoietin), or where analyte is assessed following such treatment and the concentration or the amount of analyte is compared against a predetermined level. An unfavorable concentration of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

It goes without saying that, while certain embodiments herein are advantageous when employed to assess sepsis, or risk of sepsis, the assays and kits also optionally can be employed to assess angiopoietin in other diseases, disorders and conditions as appropriate.

The method of assay also can be used to identify a compound that ameliorates sepsis. For example, a cell that expresses angiopoietin can be contacted with a candidate compound. The level of expression of angiopoietin in the cell contacted with the compound can be compared to that in a control cell using the method of assay described herein.

Generally, any method that can detect or quantify biomarkers in a sample can be used in the methods described herein. These methods include physical and molecular biology methods in addition to immunological methods described previously herein. For example, suitable physical methods include mass spectrometric methods, fluorescence resonance energy transfer (FRET) assays, chromatographic assays, and dye-detection assays. Suitable molecular biology methods that can be used include, but are not limited to, Northern or Southern blot hybridization, nucleic acid dot- or slot-blot hybridization, in situ hybridization, nucleic acid chip assays, PCR, reverse transcriptase PCR (RT-PCR), or real time PCR (taq-man PCR). Other methods to detect biomarkers include, e.g., nuclear magnetic resonance (NMR), fluorometry, colorimetry, radiometry, luminometry, or other spectrometric methods, plasmon-resonance (e.g. BIACORE), and one- or two-dimensional gel electrophoresis.

Once measured, the concentration of angiopoietin-1 and that of any other additional biomarker being assessed is compared to a predetermined reference value for the specific biomarker. A measured, i.e. determined, angiopoietin-1 concentration that exceeds the reference angiopoietin-1 value is indicative of sepsis or increased risk of sepsis in the subject. The reference value may be determined in one of several ways. For example, the angiopoietin-1 reference value can be the angiopoietin-1 concentration measured in a sample taken from a control subject, or may be the median angiopoietin-1 concentration calculated from the concentrations measured in multiple control samples taken from a group of control subjects. A median angiopoietin-1 concentration is preferably obtained from a group of at least 20 control subjects, at least 30 control subjects, or at least 40 control subjects. The predetermined reference value for the biomarker can be a predetermined cutoff value or predetermined level.

A “control subject” is a healthy subject, i.e. a subject having no clinical signs or symptoms of sepsis. Preferably a control subject is clinically evaluated for otherwise undetected signs or symptoms of sepsis, which evaluation may include routine laboratory testing.

Alternatively, an angiopoietin-1 cutoff value (i.e., predetermined cutoff value or predetermined level) can be determined by a receiver operating curve (ROC) analysis from biological samples of a patient group. ROC analysis as generally well known in the biological arts is a determination of the ability of a test to discriminate one condition from another, e.g. diseased cases from normal cases, or to compare the diagnostic performance of two or more laboratory or diagnostic tests. A description of ROC analysis as applied according to the present disclosure is provided in P. J. Heagerty et al., Time-dependent ROC curves for censored survival data and a diagnostic marker, Biometrics 56:337-44 (2000), the disclosure of which is hereby incorporated by reference in its entirety. Alternatively, an angiopoietin-1 cutoff value can be determined by a quartile analysis of biological samples of a patient group. For example, an angiopoietin-1 cutoff value can be determined by selecting a value that corresponds to any value in the 25th-75th percentile range, preferably a value that corresponds to the 25th percentile, the 50th percentile or the 75th percentile, and more preferably the 75th percentile. In one aspect, the angiopoietin-1 reference value obtained from the median of a relevant patient group is about 20 ng/ml in serum. In another aspect, an angiopoietin-1 reference value obtained from quartile analysis at the 75th percentile is about 28 ng/ml in serum.

The method may further include assessing at least one additional biomarker of sepsis, for example by measuring the concentration at least one additional biomarker in the biological sample, and comparing the measured concentration to a reference value (i.e., predetermined cutoff value or predetermined level) for each additional biomarker being assessed. One, two, three, four or more additional biomarkers may be assessed. Additional such biomarkers of sepsis include, but are not limited to, angiopoietin-2, procalcitonin, CRP, TNF, IL-1, IL-6, and G-CSF. A reference value may be similarly determined for any other biomarker of sepsis, as described herein with respect to determining a reference value for angiopoietin-1. Typically, a measured i.e., determined concentration of any additional biomarker in a biological sample that exceeds the reference value for that biomarker is also indicative of sepsis or increased risk of sepsis in the subject. Instances of biomarkers for which the opposite is true are nevertheless possible, i.e. biomarkers for which the relationship between concentration in a biological sample and instance of sepsis or increased risk of sepsis is inverse, such that a determined biomarker concentration that is below the reference value for the biomarker is indicative of sepsis or increased risk of sepsis in the subject.

For example, elevated levels procalcitonin in the blood have been used as diagnostic biomarkers of sepsis. Procalcitonin is a peptide precursor of the hormone calcitonin. Calcitonin is involved with calcium homeostasis. Procalcitonin is composed of 116 amino acids and is produced by parafollicular cells (C cells) of the thyroid and by the neuroendocrine cells of the lung and the intestine. Plasma or serum concentrations of procalcitonin and angiopoietin-2 are increased in patients with sepsis. Accordingly, the concentrations of both angiopoietin-1 and procalcitonin or angiopoietin-2 may be determined and each compared to a corresponding predetermined reference value as described herein. A suitable reference value for procalcitonin is, for example, a median of about 0.05 ng/ml in serum. Similarly, the concentrations of both angiopoietin-1 and angiopoietin-2 may be determined and each compared to a corresponding predetermined reference value as described herein.

### C. KITS

Provided herein are kits which may be used for diagnosing a subject suffering from sepsis or at increased risk of developing sepsis as described previously herein.

Kits can containing instructions for their use. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

As mentioned previously, provided herein is a kit for assaying a test sample for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof). The kit can comprise at least one component for assaying the test sample for angiopoietin and instructions for assaying the test sample for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof). The at least one component includes at least one composition comprising an isolated antibody that specifically binds to angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof). The antibody has a variably heavy domain region and a variable light domain region. The antibody can optionally labeled with a detectable label.

For example, the kit can comprise instructions for assaying the test sample for angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof) by immunoassay, e.g., chemiluminescent microparticle immunoassay. The instructions can be in paper form or computer-readable form, such as a disk, CD, DVD, or the like. The antibody can be an angiopoietin capture antibody and/or an angiopoietin detection antibody. Alternatively or additionally, the kit can comprise a calibrator or control, e.g., purified, and optionally lyophilized, angiopoietin (such as angiopoietin-1, angiopoietin-2, fragments of angiopoietin-1, fragments of angiopoietin-2, variants of angiopoietin (angiopoietin-1 or soluble angiopoietin-2) or any combinations thereof), and/or at least one container (e.g., tube, microtiter plates or strips, which can be already coated with an anti-angiopoietin monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying angiopoietin.

Any antibodies, which are provided in the kit, such as recombinant antibodies specific for angiopoietin, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of sepsis. Examples of analytes include, but are not limited to angiopoietin-1, angiopoietin-2 and procalcitonin, CRP, TNF, IL-1, IL-6, and G-CSF as well as other analytes and biomarkers discussed herein. It can be preferred that one or more components for assaying a test sample for angiopoietin enable the determination of the presence, amount or concentration of angiopoietin-1. A sample, such as a urine sample or serum sample, can be assayed for angiopoietin-1 using TOF-MS and an internal standard.

The kit (or components thereof), as well as the method of determining the concentration of angiopoietin in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing angiopoietin, such as angipoietin-1, is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, angiopoietin, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte angiopoietin in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an I-STAT® cartridge format, using a nucleic acid sequence linked to the signal antibody as a signal amplifier.

If desired, multiple concentrations of each antibody can be included in the kit to facilitate the generation of a standard curve to which the signal detected in the test sample can be compared. Alternatively, a standard curve can be generated by preparing dilutions of a single antibody solution provided in the kit.

It will be readily apparent to those skilled in the art that other suitable modifications and adaptations of the methods of the present disclosure described herein are obvious and may be made using suitable equivalents without departing from the scope of the present disclosure or the embodiments disclosed herein. Having now described the present disclosure in detail, the same will be more clearly understood by reference to the following example which is included for purposes of illustration only and not intended to limit the scope of the present disclosure. The disclosures of all journal references, U.S. patents and publications referred to herein are hereby incorporated by reference in their entireties.

The following examples are provided for illustrative purposes, and are not intended to be limiting.

### EXAMPLES

### Example 1

### Measurement of Biomarkers

Thirty sepsis specimens were purchased from CRCCC (Clinical Research Center of Cape Cod). Twenty-eight normal specimens were purchased by ProMedDx. For each specimen, Angiopoietin-1 protein concentration was determined using a Angiopoietin-1 ELISA (Enzyme-Linked Immuno Sorbent Assay) kit (Research Use Only) from R&D Systems Co., Ltd. (Minneapolis, USA), according to manufacturer instructions. Angiopoietin-2 protein concentration was determined using a Angiopoietin-2 ELISA (Enzyme-Linked Immuno Sorbent Assay) kit (Research Use Only) from R&D Systems Co., Ltd. (Minneapolis, USA), according to manufacturer instructions. The assays were conducted utilizing two-step sandwich assays.

Procalcitonin protein concentration was determined using a Procalcitonin automated immunoassay ECLusys reagent (Regulatory approval in Japan) from Roche Brahms Co., Ltd. (Basel, Switzerland) and Procalcitonin ELISA (Enzyme-Linked Immuno Sorbent Assay) kit (Research Use Only) from Ray Biotech Co., Ltd. (Georgia, USA), according to manufacturer instructions.

Results

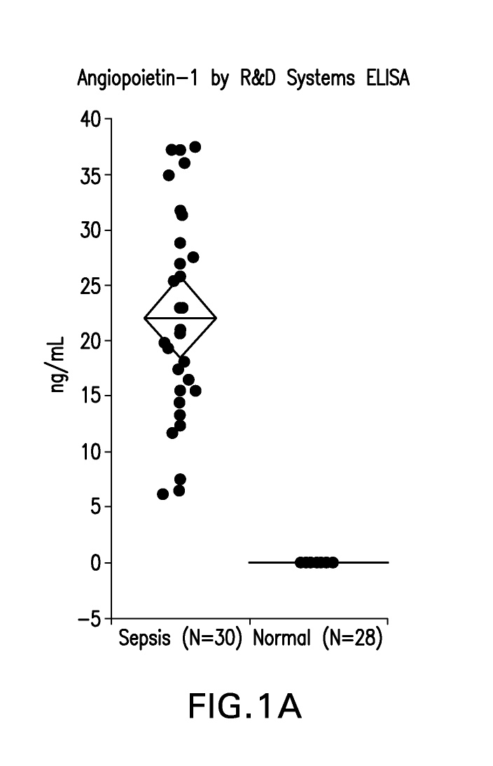

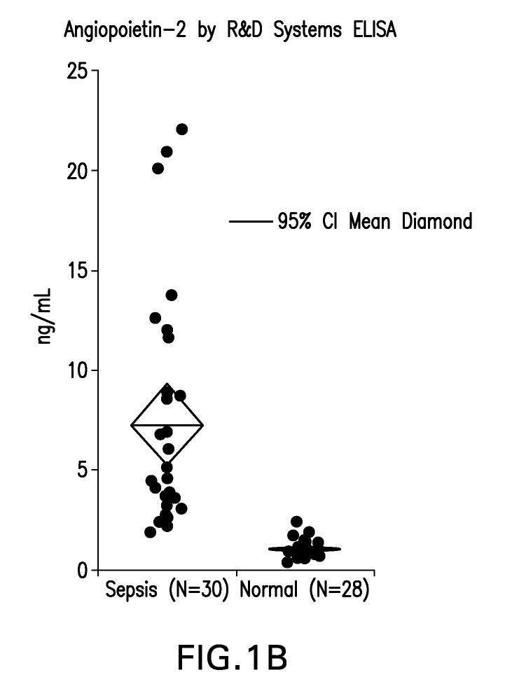

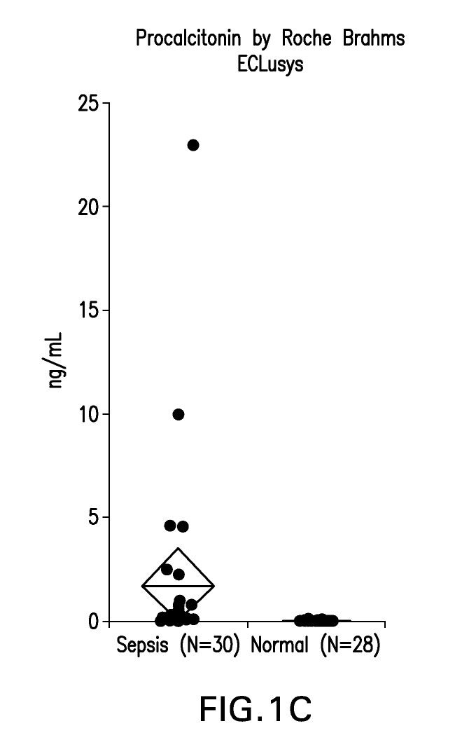

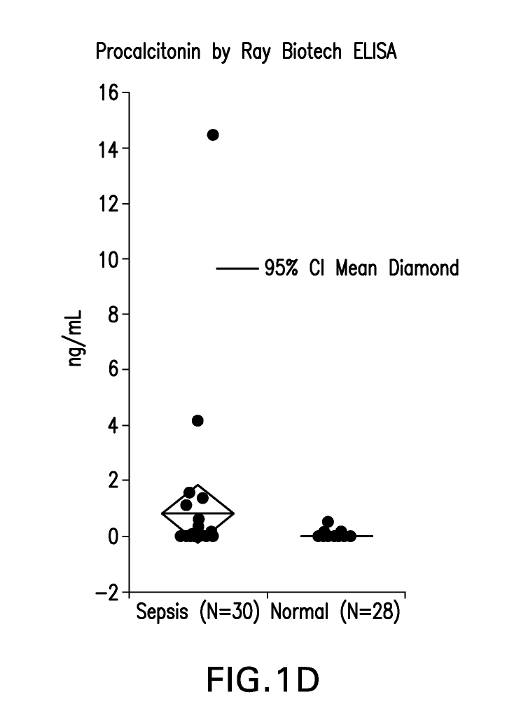

FIG. 1 shows scatter plots of individual Procalcitonin by Roche Brahms ECLusys, Angiopoietin-1, Angiopoietin-2 protein levels measured in the serum from sepsis and normal individuals. Raw Data is presented in Appendix 1. A 95% CI (Confidence Interval) Mean Diamond in each graph was calculated by the measured values of specimens. The 95% confidence interval mean shows that true mean value about the population exists within the confidence interval at the frequency of 95%. The horizontal line shows the mean of measured values of specimens. As shown in FIG. 1A, the mean Angiopoietin-1 level in sepsis subjects was substantially higher than in the normal subjects.

The values of angiopoietin-2 in sepsis specimens were also found to be increased significantly when they were compared to the values in normal specimens (FIG. 1B).

FIG. 1C shows the results of Procalcitonin by Roche Brahms ECLusys, which is a regulatory approved marker for sepsis in Japan.

FIG. 1D shows the results of Procalcitonin by the ELISA of Ray Biotech Co., Ltd.

Using a cut off of 0.05 ng/mL, 77% (23/30) of sepsis subjects showed elevated levels of Procalcitonin above the cutoff. The detection of sepsis in subjects by using Angiopoietin-1 was thus superior than detection of sepsis using Procalcitonin.

### Example 2

### Low False Positive Rate and Low False Negative Rate of Angiopoietin-1 Serum Levels in Detecting Sepsis

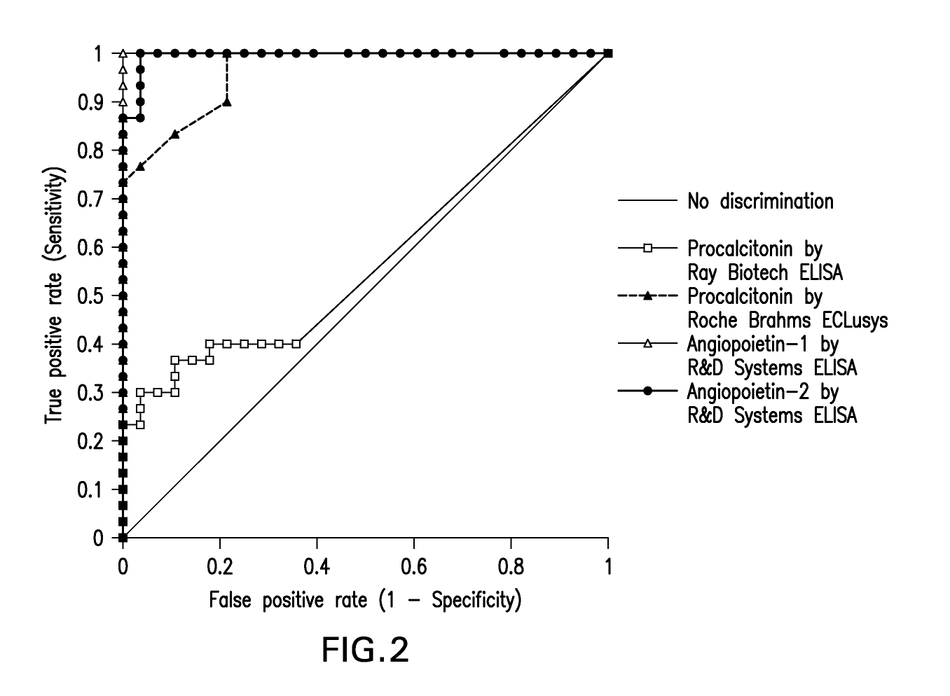

A Receiver Operating Characteristic (ROC) plot was generated of the observed true positive rate of the elevated four markers levels in sepsis subjects against the observed false positive rate of the elevated four markers levels in normal subjects (FIG. 2). The diagonal line across the plot indicates the plot expected from the worst possible prediction method in which marker levels would not discriminate at all between sepsis subjects and normal subjects. A best possible prediction method is expected to yield a point in the upper left corner or coordinate (0,1) of the ROC space, representing 100% sensitivity (no false negatives) and 100% specificity (no false positives). Thus, an area under the curve (AUC) derived from a plot of actual data which approaches the value of 1.0 represents a best possible prediction method.

TABLE 1

| Test | Area | 95% CI | SE |
|---|---|---|---|
| Procalcitonin by Ray Biotech ELISA | 0.577 | 0.446 0.709 | 0.067 |
| Procalcitonin by Roche Brahms ECLusys | 0.963 | 0.924 1.000 | 0.020 |
| Angiopoietin-1 by R&D Systems ELISA | 1.000 | - to - | 0.000 |
| Angiopoietin-2 by R&D Systems ELISA | 0.995 | 0.985 1.000 | 0.005 |

As can be seen in FIG. 2 and Table 1, the AUC of Angiopoietin-1 is 1.000, indicating that elevated Angiopoietin-1 level (i.e. a value of Angiopoietin-1 protein above the cutoff (5 ng/mL) is a very strong predictive classifier for sepsis.

For Procalcitonin by Roche Brahms ECLusys, the AUC is 0.963, indicating that the performances of predictive classifier for sepsis by Procalcitonin are worse than these by Angiopoietin-1.

Elevated levels of angiopoietin-1 in serum can be useful for discrimination between sepsis specimens and normal specimens.

### Example 3

### Additional Measurements of Angiopoietin-1 and Angiopoietin-2 Concentrations

Seventeen (17) serum specimens from subjects diagnosed with sepsis were obtained from Bioreclamation Co., Ltd. (New York, USA). Fifteen (15) serum specimens from subjects diagnosed with sepsis were obtained from Discovery Life Sciences Co., Ltd. (California, USA). For each specimen, Angiopoeitin-1 protein concentration was determined using an Angiopoietin-1 ELISA kit from R&D Systems Co., Ltd. (Minneapolis, USA) and Ray Biotech Co., Ltd. (Georgia, USA). Angiopoietin-2 protein concentration was determined using an Angiopoietin-2 ELISA kit from R&D Systems Co., Ltd. and ray Biotech Co., Ltd.

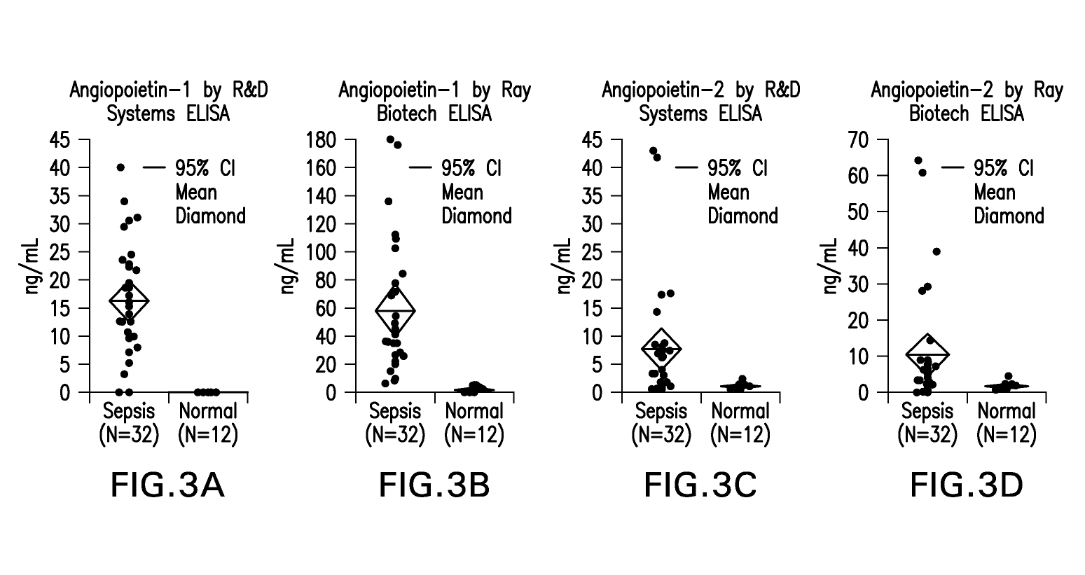

In particular, FIG. 3 shows a scatter plot of individual values of Angiopoietin-1 and Angiopoietin-2. As illustrated, elevation of Angiopoietin-1 and Angiopoietin-2 in sepsis specimens was confirmed by the specimens purchased by two other vendors (Bioreclamation and Disccovery Life Sciences Co., Ltd.) and two ELISA kits (R&D Systems and Ray Biotech). The values of Angiopoietin-1 and Angiopoietin-2 by the Ray Biotech ELISA kit were higher than those obtained by the R&D Systems ELISA kit.

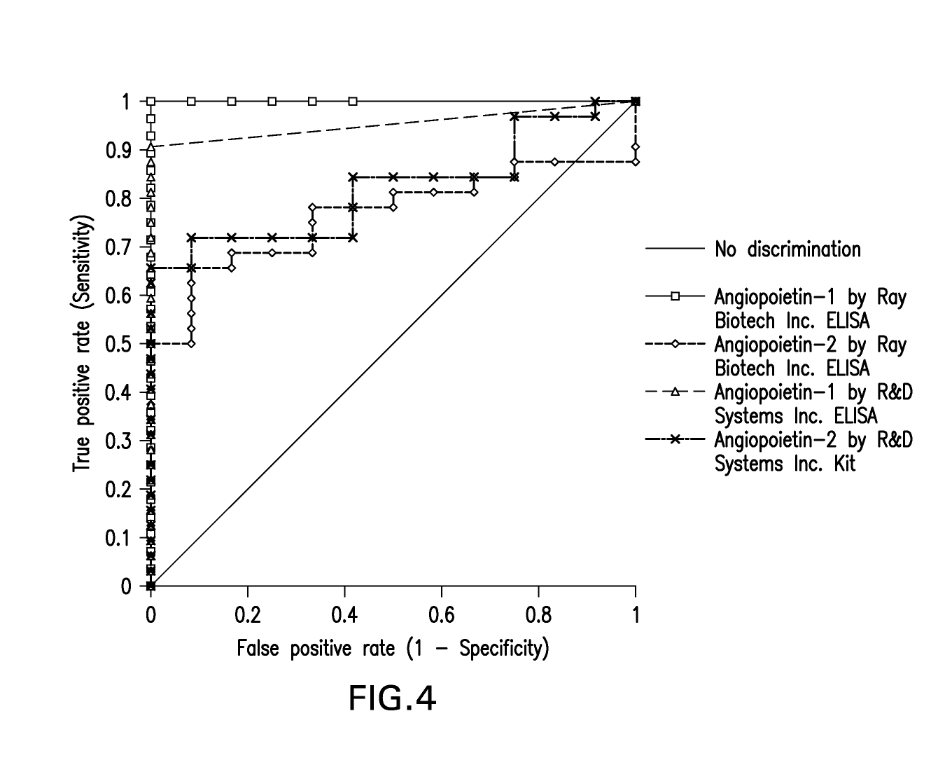

FIG. 4 shows a Receiver Operating Characteristic (ROC) plot of Angiopoietin-1 and Angiopoietin-2 by the R&D Systems ELISA kit and the Ray Biotech ELISA kit. In particular, the figure shows the plot of the observed, true positive rate of the elevated four marker levels in subjects with sepsis versus the observed false positive rate of the elevated four marker levels of normal subjects. The diagonal line across the plot indicates the plot that was expected from the worst possible prediction method in which marker levels would not discriminate between subjects with sepsis and normal subjects. A best possible prediction method is expected to yield a point in the upper left corner or coordinate (0,1) of the ROC space, representing 100% sensitivity (no false negatives) and 100% specificity (no false positives). Thus, an area under the curve (AUC) derived from a plot of actual data which approaches the value of 1.0 represents a best possible prediction method. As can be seen in FIG. 4 and Table 2, the AUC of Angiopoietin-1 by Ray Biotech ELISA kit is 1.00 and the AUC of Angiopoietin-1 by R&D Systems ELISA kit is 0.95, indicating that elevated Angiopoietin-1 level is a very strong predictive classifier for sepsis. By the Ray Biotech ELISA kit, the AUC of Angiopoietin-2 is 0.77, and the AUC of Angiopoietin-2 by the R&D Systems ELISA kit is 0.82, indicating that the performances of predictive classifiers for sepsis by Angiopoietin-2 are worse than these by Angiopoietin-1. As noted above, elevated levels of angiopoietin-1 in serum can be useful in discriminating between sepsis specimens and normal specimens.

TABLE 2

| Disease | n |
|---|---|
| Normal | 12 |
| Sepsis | 32 |

| Test | Area | 95% CI | SE |
|---|---|---|---|
| Angiopoietin-1 by Ray Biotech Inc. ELISA | 1.00 | - to - | 0.000 |
| Angiopoietin-2 by Ray Biotech Inc. ELISA | 0.77 | 0.63 to 0.90 | 0.071 |
| Angiopoietin-1 by R&D Systems Inc. ELISA | 0.95 | 0.90 to 1.00 | 0.026 |
| Angiopoietin-2 by R&D Systems Inc. Kit | 0.82 | 0.70 to 0.94 | 0.062 |

### REFERENCES

Angiopoietin-1 and angiopoietin-2 as clinically informative prognostic biomarkers of morbidity and mortality in severe sepsis Crit. Care Med. 2011 April; 39(4):702-710
Angiopoietin Balance in Septic Shock Patients Treated by Direct Hemoperfusion with Polymyxin B-immobilized Fiber. Ther Apher Dial. 13, page 520-527, 2009
Does low angiopoietin-1 predict adverse outcome in sepsis?Crit. Care. 2010; 14(4):180. Epub 2010 Jul. 20
Excess circulating angiopoietin-2 is a strong predictor of mortality in critically ill medical patients. Crit. Care Vol. 12, No. 6, R147, 2008
Elevated serum angiopoietin-2 levels are associated with increased mortality in Sepsis. Shock, Vol. 31, No. 4, page 348-353, 2009
Endothelial activation, dysfunction and permeability during severe infections. Curr Opin Hematol. 2011 May; 18(3):191-6.

APPENDIX 1
Rep. = 1

| | | Serum markers | | | | | | | |
|---|---|---|---|---|---|---|---|---|---|
| | | | | Procalcitonin | | | | | |
| | | Procalcitonin (ELISA) | | ECLusys | | Angiopoietin-1 | | Angiopoietin-2 | |
| | | Manufacturers | | | | | | | |
| | | Ray Biotech | | Roche Brahms | | R&D Systems | | R&D Systems | |
| | | Measurable range | | | | | | | |
| | | 27.43-20.000 pg/mL | | 0.02-100 ng/mL | | 62.5-4000 pg/mL | | 46.9-3000 pg/mL | |
| | | Sample dilution factor | | | | | | | |
| Vendors | Sample ID | ×1 | | ×1 | | ×50 | | ×5 | |
| CRCCC - | 2676001-SepA01 | 4.15 | ng/mL | 22.95 | ng/mL | 14.44 | ng/mL | 5.15 | ng/mL |
| Sepsis | 2676001-SepA02 | 0.37 | ng/mL | 0.97 | ng/mL | 16.46 | ng/mL | 12.66 | ng/mL |
| | 2676001-SepA03 | <0.027 | ng/mL | 0.25 | ng/mL | 20.68 | ng/mL | 3.23 | ng/mL |
| | 2676001-SepA04 | <0.027 | ng/mL | 0.03 | ng/mL | 11.67 | ng/mL | 2.37 | ng/mL |
| | 2676001-SepA05 | <0.027 | ng/mL | 0.70 | ng/mL | 20.98 | ng/mL | 3.67 | ng/mL |
| | 2676001-SepA06 | <0.027 | ng/mL | 0.03 | ng/mL | 26.95 | ng/mL | 2.20 | ng/mL |
| | 2676001-SepA07 | <0.027 | ng/mL | 0.14 | ng/mL | 37.19 | ng/mL | 2.75 | ng/mL |
| | 2676001-SepA08 | 0.08 | ng/mL | 0.79 | ng/mL | 31.67 | ng/mL | 5.05 | ng/mL |
| | 2676001-SepA09 | <0.027 | ng/mL | 0.19 | ng/mL | 13.24 | ng/mL | 6.81 | ng/mL |
| | 2676001-SepA10 | <0.027 | ng/mL | 0.02 | ng/mL | 25.81 | ng/mL | 2.57 | ng/mL |
| | 2676001-SepA11 | <0.027 | ng/mL | 0.12 | ng/mL | 36.03 | ng/mL | 4.13 | ng/mL |
| | 2676001-SepA12 | 0.60 | ng/mL | 2.48 | ng/mL | 7.48 | ng/mL | 12.04 | ng/mL |
| | 2676001-SepA13 | 0.11 | ng/mL | 0.84 | ng/mL | 27.48 | ng/mL | 8.73 | ng/mL |
| | 2676001-SepA14 | 1.11 | ng/mL | 4.59 | ng/mL | 6.14 | ng/mL | 11.66 | ng/mL |
| | 2676001-SepA15 | <0.027 | ng/mL | 0.04 | ng/mL | 22.94 | ng/mL | 3.60 | ng/mL |
| | 2676001-SepA16 | 14.43 | ng/mL | 0.10 | ng/mL | 28.77 | ng/mL | 6.07 | ng/mL |
| | 2676001-SepA17 | 1.55 | ng/mL | 9.98 | ng/mL | 12.34 | ng/mL | 21.00 | ng/mL |
| | 2676001-SepA18 | 0.00 | ng/mL | 0.28 | ng/mL | 22.94 | ng/mL | 8.89 | ng/mL |
| | 2676001-SepA19 | 1.38 | ng/mL | 4.55 | ng/mL | 25.36 | ng/mL | 20.17 | ng/mL |
| | 2676001-SepA20 | <0.027 | ng/mL | 0.22 | ng/mL | 18.04 | ng/mL | 8.60 | ng/mL |
| | 2676001-SepA21 | <0.027 | ng/mL | 0.02 | ng/mL | 37.19 | ng/mL | 4.44 | ng/mL |
| | 2676001-SepA22 | <0.027 | ng/mL | 0.27 | ng/mL | 15.49 | ng/mL | 6.90 | ng/mL |
| | 2676001-SepA23 | <0.027 | ng/mL | 0.13 | ng/mL | 6.44 | ng/mL | 13.77 | ng/mL |
| | 2676001-SepA24 | 0.18 | ng/mL | 0.04 | ng/mL | 31.29 | ng/mL | 4.59 | ng/mL |
| | 2676001-SepA25 | 0.06 | ng/mL | 0.07 | ng/mL | 37.42 | ng/mL | 2.73 | ng/mL |
| | 2676001-SepA26 | <0.027 | ng/mL | 0.02 | ng/mL | 15.49 | ng/mL | 1.89 | ng/mL |
| | 2676001-SepA27 | <0.027 | ng/mL | 0.45 | ng/mL | 19.77 | ng/mL | 2.19 | ng/mL |
| | 2676001-SepA28 | 0.55 | ng/mL | 2.23 | ng/mL | 17.44 | ng/mL | 22.13 | ng/mL |
| | 2676001-SepA29 | <0.027 | ng/mL | 0.13 | ng/mL | 34.9 | ng/mL | 3.07 | ng/mL |
| | 2676001-SepA30 | <0.027 | ng/mL | 0.06 | ng/mL | 19.3 | ng/mL | 3.86 | ng/mL |
| ProMedDx - | 2279002-Nor31 | <0.027 | ng/mL | <0.02 | ng/mL | <3.1 | ng/mL | 1.02 | ng/mL |
| Normal | 2279002-Nor32 | <0.027 | ng/mL | <0.02 | ng/mL | <3.1 | ng/mL | 1.74 | ng/mL |
| | 2279002-Nor33 | <0.027 | ng/mL | <0.02 | ng/mL | <3.1 | ng/mL | 1.12 | ng/mL |
| | 2279002-Nor34 | 0.04 | ng/mL | 0.04 | ng/mL | <3.1 | ng/mL | 1.35 | ng/mL |
| | 2279002-Nor35 | <0.027 | ng/mL | 0.06 | ng/mL | <3.1 | ng/mL | 1.28 | ng/mL |
| | 2279002-Nor37 | <0.027 | ng/mL | <0.02 | ng/mL | <3.1 | ng/mL | 0.56 | ng/mL |
| | 2279002-Nor39 | <0.027 | ng/mL | <0.02 | ng/mL | <3.1 | ng/mL | 1.88 | ng/mL |
| | 2279002-Nor41 | 0.17 | ng/mL | <0.02 | ng/mL | <3.1 | ng/mL | 1.36 | ng/mL |
| | 2279002-Nor43 | 0.04 | ng/mL | <0.02 | ng/mL | <3.1 | ng/mL | 1.02 | ng/mL |
| | 2279002-Nor44 | 0.06 | ng/mL | <0.02 | ng/mL | <3.1 | ng/mL | 0.77 | ng/mL |
| | 2279002-Nor49 | <0.027 | ng/mL | <0.02 | ng/mL | <3.1 | ng/mL | 1.40 | ng/mL |
| | 2279002-Nor51 | <0.027 | ng/mL | <0.02 | ng/mL | <3.1 | ng/mL | 1.15 | ng/mL |
| | 2279002-Nor54 | 0.52 | ng/mL | <0.02 | ng/mL | <3.1 | ng/mL | 1.47 | ng/mL |
| | 2279002-Nor58 | <0.027 | ng/mL | <0.02 | ng/mL | <3.1 | ng/mL | 0.83 | ng/mL |
| | 2279002-Nor60 | <0.027 | ng/mL | <0.02 | ng/mL | <3.1 | ng/mL | 0.92 | ng/mL |
| | 2279002-Nor61 | 0.06 | ng/mL | 0.03 | ng/mL | <3.1 | ng/mL | 0.87 | ng/mL |
| | 2279002-Nor63 | <0.027 | ng/mL | 0.03 | ng/mL | <3.1 | ng/mL | 2.40 | ng/mL |
| | 2279002-Nor64 | <0.027 | ng/mL | <0.02 | ng/mL | <3.1 | ng/mL | 0.70 | ng/mL |
| | 2279002-Nor67 | 0.17 | ng/mL | 0.04 | ng/mL | <3.1 | ng/mL | 0.88 | ng/mL |
| | 2279002-Nor68 | 0.07 | ng/mL | <0.02 | ng/mL | <3.1 | ng/mL | 0.70 | ng/mL |
| | 2279002-Nor69 | <0.027 | ng/mL | <0.02 | ng/mL | <3.1 | ng/mL | 1.20 | ng/mL |
| | 2279002-Nor73 | <0.027 | ng/mL | <0.02 | ng/mL | <3.1 | ng/mL | 0.58 | ng/mL |
| | 2279002-Nor78 | <0.027 | ng/mL | <0.02 | ng/mL | <3.1 | ng/mL | 0.97 | ng/mL |
| | 2279002-Nor82 | <0.027 | ng/mL | <0.02 | ng/mL | <3.1 | ng/mL | 0.70 | ng/mL |
| | 2279002-Nor84 | <0.027 | ng/mL | <0.02 | ng/mL | <3.1 | ng/mL | 0.64 | ng/mL |
| | 2279002-Nor86 | 0.03 | ng/mL | <0.02 | ng/mL | <3.1 | ng/mL | 0.60 | ng/mL |
| | 2279002-Nor87 | 0.05 | ng/mL | <0.02 | ng/mL | <3.1 | ng/mL | 0.35 | ng/mL |
| | 2279002-Nor88 | <0.027 | ng/mL | 0.03 | ng/mL | <3.1 | ng/mL | 0.84 | ng/mL |

What is claimed is:

1. A method for providing a diagnosis, prognosis or risk classification of a subject having or at risk of having sepsis, the method comprising
a. determining the concentration of angiopoietin-1 in a biological sample of a subject; and
b. comparing the determined angiopoietin-1 concentration with a reference angiopoietin-1 value, wherein a determined angiopoietin-1 concentration of the subject greater than the reference value is indicative of sepsis or increased risk of sepsis.

2. The method of claim 1, further comprising assessing at least one additional biomarker of sepsis.

3. The method of claim 2, wherein the additional biomarker of sepsis is selected from angiopoietin-2 and procalcitonin.

4. The method of claim 1, 2 or 3, wherein the reference angiopoietin-1 value is an angiopoietin-1 value of a control sample, or an angiopoietin-1 cutoff value.

5. The method of claim 4, wherein the reference angiopoietin-1 value is the angiopoietin-1 value of a control sample selected from a biological sample of a control subject and an angiopoietin-1 standard.

6. The method of claim 4, wherein the reference angiopoietin-1 value is the angiopoietin-1 value of a control sample and is a median angiopoietin-1 concentration of a plurality of control samples from a group of control subjects.

7. The method of claim 6, wherein the determined angiopoietin-1 concentration of the subject is at least double than the reference value.

8. The method of claim 4, wherein the reference angiopoietin-1 value is the angiopoietin-1 cutoff value determined by a receiver operating curve (ROC) analysis from biological samples of a patient group.

9. The method of claim 4, wherein the reference angiopoietin-1 value is the angiopoietin-1 cutoff value determined by a quartile analysis of biological samples of a patient group.

10. The method of claim 4, wherein the reference angiopoietin-1 value is the angiopoietin-1 cutoff value, and is about 5 ng/mL in serum.

11. The method of any preceding claim, wherein the angiopoietin-1 concentration is the angiopoietin-1 serum concentration.

12. The method of any preceding claim, wherein the method comprises providing a diagnosis of sepsis.

13. The method of any preceding claim, wherein the method comprises providing a prognosis selected from determining the severity of sepsis and risk assessment of the subject with sepsis.

14. The method of any preceding claim, wherein the subject is a human.

15. The method of claim 5, wherein the reference angiopoietin-1 value is the biological sample of a control subject, and wherein the control subject is a human.

16. The method of any preceding claim, wherein the biological sample of a subject is selected from a bodily fluid, whole blood, plasma, serum, urine and a cell culture suspension or fraction thereof.

17. The method of any preceding claim, wherein the biological sample of a subject is blood plasma or blood serum.

18. The method of any preceding claim, wherein determining the concentration of angiopoietin-1 comprises an immunological method with molecules binding to the angiopoietin-1.

19. A method for the diagnosis, prognosis and/or risk stratification of sepsis in a subject having or suspected of having sepsis, the method comprising detecting an increased angiopoietin-1 concentration in the subject relative to a control subject not having sepsis.

20. A kit for performing the method of any preceding claim, the kit comprising:
a. at least one reagent capable of specifically binding angiopoietin-1 to quantify the angiopoietin-1 concentration in the biological sample of a subject; and
b. a reference standard indicating a reference angiopoietin-1 concentration.

21. The kit of claim 20, further comprising at least one additional reagent capable of binding at least one additional biomarker of sepsis in the biological sample to quantify the concentration of the at least one additional biomarker in the biological sample, and a reference standard indicating a reference concentration of the at least one additional biomarker of sepsis in the biological sample.

22. The kit of claim 20, wherein the at least one reagent comprises at least one antibody capable of specifically binding angiopoietin-1.
